(19) United States
(12) Patent Application Publication
(10) Pub. No.: US 2025/0367116 A1
(43) Pub. Date: Dec. 4, 2025

(54) NITRIC OXIDE TRANSPORTER FOR PREVENTING OR TREATING PULMONARY ARTERIAL HYPERTENSION AND METHOD FOR MANUFACTURING THE SAME

(71) Applicant: INDUSTRY-ACADEMIC COOPERATION FOUNDATION, YONSEI UNIVERSITY, Seoul (KR)

(72) Inventors: Jin Kee HONG, Seoul (KR); Se Yong JUNG, Seoul (KR); Yoo Gyeong OH, Seoul (KR)

(21) Appl. No.: 19/185,822

(22) Filed: Apr. 22, 2025

(30) Foreign Application Priority Data
Jun. 4, 2024 (KR) 10-2024-0073083

(51) Int. Cl. A61K 9/113; A61K 9/00; A61K 33/00; A61P 9/12

(52) U.S. Cl. CPC A61K 9/113; A61K 9/0078; A61K 33/00; A61P 9/12

(57) ABSTRACT

An embodiment relates to a method of preparing an inhalable type nitric oxide transporter capable of treating pulmonary arterial hypertension using a nebulizer, and more particularly, to a method of preparing a new type of therapeutic agent capable of treating pulmonary arterial hypertension more effectively by allowing particles having a low density due to high porosity of the particles to reach lesions deep in the lungs through a nebulizer. The inhalable type nitric oxide transporter of the embodiment has been confirmed to have effects such as inducing vasodilation, enhancing nitric oxide delivery efficiency, and inhibiting phagocytosis, and can be utilized as a therapeutic agent for pulmonary arterial hypertension.

### STATEMENT REGARDING PRIOR DISCLOSURES BY THE INVENTORS

A non-patent literature entitled, “Inhalable Nitric Oxide Delivery Systems for Pulmonary Arterial Hypertension Treatment” which was published on or around Dec. 6, 2023, is not prior art under 35 U.S.C. 102(b) as being a disclosure made directly or indirectly by the inventor or a joint inventor 1 year or less before the effective filing date of the instant application. A copy of the non-patent literature prior disclosure is being submitted with the instant application in an Information Disclosure Statement pursuant to 37 CFR 1.97 and 1.98.

### CROSS REFERENCE TO RELATED APPLICATION

The present application claims priority to Korean Patent Application No. 10-2024-0073083, filed Jun. 4, 2024, the entire contents of which are incorporated here for all purposes by this reference.

### BACKGROUND OF THE INVENTION

### Field of the Invention

The present invention relates to an inhalable type nitric oxide transporter capable of treating pulmonary arterial hypertension using a nebulizer and a method of preparing the same. Specifically, the present invention relates to a new type of therapeutic agent capable of treating pulmonary arterial hypertension more effectively by allowing particles having a low density due to high porosity of the particles to reach lesions deep in the lungs through a nebulizer, and a method of preparing the same.

### Description of the Related Art

Nitric oxide (NO) is a gaseous signaling molecule whose pharmacological effects were verified through the Nobel Prize in Physiology or Medicine in 1998, and has been actively studied in the fields of medicine and engineering. In particular, NO has been in the spotlight in the treatment of pulmonary arterial hypertension as a powerful therapeutic substance because it is capable of expanding blood vessels by increasing the expression of cyclic guanosine monophosphate (cyclic GMP (cGMP)) in vascular smooth muscle cells (SMC). Unlike chemical drugs, gaseous NO is easy to deliver transpulmonarily, and thus is used as an inhalable type therapeutic substance. However, it is still a very challenging task to stably deliver radical-type NO, which has a short half-life of two to three seconds and a limited diffusion distance of less than 100 μm, to a target site through inhalation (A dv. Healthcare Mater, 11, 2102095 (2022)).

Pulmonary arterial hypertension is a rare intractable disease with a high mortality rate that may cause right ventricular failure and sudden cardiac death due to increased pressure and pulmonary vascular resistance in the pulmonary artery, which is a blood vessel that supplies blood from the heart to the lungs. The molecular biological mechanisms of pulmonary arterial hypertension include the endothelial pathway, the NO pathway, and the prostacyclin pathway. Selective therapeutic agents for pulmonary arterial hypertension have been developed for the mechanisms, and combined therapies thereof are being used.

Oral drugs (riociguat, sildenafil, etc.) targeting the NO pathway, which is the most important and powerful mechanism, have been developed, but there is a risk of side effects due to systemic vasodilation. In contrast, NO may act directly only on pulmonary arterial hypertension lesions, so it is possible to induce selective vasodilation. However, its use is very limited up to date because it may be applied only to patients who are able to breathe through tracheal intubation. In addition, information on the concentration and release behavior of NO reaching the lesion is not available, and therefore, a complex delivery device is required.

Against this background, the present inventors overcame the limitations of the prior art and prepared an inhalable type NO transporter by controlling the porous structure of the particles. The water inflow generated during particle preparation was controlled depending on the molecular weight of the NO donor to vary the size of the formed pores, and the NO release behavior was controlled so that NO necessary for vasodilation is continuously released.

The present inventors intended to implement a technology that can deliver NO simply and stably and to prepare an inhalable type NO transporter capable of continuously releasing NO while delivering it deep into the lungs through a nebulizer by controlling the porous structure of the NO transporter. It was confirmed that using the inhalable type NO transporter has the effects of continuously releasing NO while delivering it deep into the lungs through a nebulizer, inducing vasodilation, and inhibiting phagocytosis, and the possibility of using it as a therapeutic agent for pulmonary arterial hypertension was confirmed.

### SUMMARY OF THE INVENTION

One object of the present invention is to provide an inhalable type nitric oxide (NO) transporter including a biodegradable polymer shell including an NO donor therein.

In addition, another object of the present invention is to provide a method of preparing an inhalable type NO transporter, including: (a) a step of dissolving an NO donor in an aqueous solution to prepare a first aqueous phase (W1); (b) a step of dissolving a biodegradable polymer in an organic solvent to prepare an oil phase (O); (c) a step of mixing and dispersing the first aqueous phase (W1) and the oil phase (O) to prepare a W1/O emulsion; (d) a step of mixing and dispersing the W1/O emulsion in a second aqueous phase (W2) including a surfactant to prepare a W1/O/W2 emulsion; (e) a step of stirring the W1/O/W2 emulsion to evaporate the organic solvent and then remove the surfactant and residual substances; and (f) a step of obtaining an NO transporter by freeze-drying after the removing step.

In addition, still another object of the present invention is to provide a pharmaceutical composition for preventing or treating pulmonary arterial hypertension, including the inhalable type NO transporter according to claim 1 as an active ingredient.

### BRIEF DESCRIPTION OF THE DRAWINGS

FIG. 1A shows a schematic diagram for preparing an inhalable type nitric oxide (NO) transporter having different porous structures depending on the molecular weight of branched polyethyleneimine (BPEI)/NO, which is an NO donor, and FIG. 1B shows a schematic diagram illustrating the transpulmonary delivery efficiency of the NO transporter depending on the porous structure.

FIGS. 2A-2B show a graph illustrating the results of analyzing the water inflow depending on the molecular weight of BPEI/NO and confirming the movement of water using methylene blue. FIG. 2A shows a graph illustrating the movement of methylene blue over time, and FIG. 2B shows a graph illustrating the results of quantifying the amount of methylene blue that entered poly(lactic-co-glycolic acid) (PLGA) particles.

FIG. 3 shows electron microscope images of a closed porous NO inhaler (CPNI) loaded with 25 kDa BPEI/NO and an open porous NO inhaler (OPNI) loaded with 0.8 kDa BPEI/NO.

FIG. 4 shows a graph illustrating the NO release behavior of the inhalable type NO transporter.

FIG. 5 shows fluorescence microscope images confirming the inhalable type NO transporter delivered to the lungs through a nebulizer in a respiratory tract simulation model and a graph illustrating the results of a quantitative analysis thereof.

FIG. 6 shows fluorescence microscope images analyzed after four hours of co-culture with macrophages to confirm whether the inhalable type NO transporter is removed by macrophages.

FIG. 7 shows graphs illustrating cytotoxicity analyzed after 24 hours of treatment of (a) smooth muscle cells and (b) macrophages with the inhalable type NO transporter to evaluate the biocompatibility thereof.

FIG. 8 shows the results of confirming cyclic guanosine monophosphate (cGMP) expression in smooth muscle cells by the prepared inhalable type NO transporter in vitro.

FIGS. 9A-9B show the results of confirming the anti-inflammatory effect of the prepared inhalable type NO transporter at the cellular level in vitro (macrophage). FIG. 9A shows a graph of FlowSOM analysis, and FIG. 9B shows the results of a quantitative analysis on pro-inflammatory and anti-inflammatory cells.

FIG. 10 shows the results of confirming the anti-inflammatory effect of the prepared inhalable type NO transporter at the (a) RNA and (b) protein levels in vitro (macrophage).

### DETAILED DESCRIPTION OF THE EMBODIMENTS

Hereinafter, the present invention will be described with reference to the attached drawings. However, the present invention may be implemented in various different forms and, therefore, is not limited to the embodiments described herein. When a part is said to “comprise” a certain component, unless specified otherwise, this means that it may further include other components, rather than exclude other components.

Hereinafter, the present invention will be described in more detail.

The present invention provides an inhalable type nitric oxide (NO) transporter including a biodegradable polymer shell including an NO donor therein.

In the present invention, the NO donor may be selected from the group consisting of a branched polyethyleneimine (BPEI)/NONOates complex, a pentaethylenehexamine (PEHA)/NONOates complex, and a spermine NONOates complex, and more preferably it may be a BPEI/NONOates complex.

BPEI of the BPEI/NONOates complex is a polymeric material including many amine groups and is capable of forming NONOates through a high-pressure reaction, and thus it is commonly used to synthesize an NO donor. Therefore, BPEI/NONOates (BPEI/NO) can stably release NO by the electrostatic attraction between amines and NONOates, and the strength of the attraction between amines and NONOates varies depending on the molecular weight of BPEI, so that the release behavior of NO may be controlled.

In the present invention, the biodegradable polymer may be selected from the group consisting of hyaluronic acid, gelatin, starch, chitin, cellulose, alginate, collagen, heparin, chitosan, polylactic acid (PLA), polyglycolic acid (PGA), poly(lactic-co-glycolic acid) (PLGA), polycaprolactone (PCL), polydioxanone (PDO), poly(trimethylene carbonate) (PTMC), and polyhydroxyalkanoate (PHA), and more preferably, PLGA.

In the present invention, the molecular weight of the BPEI/NONOates complex may be 0.1 to 3.0 kDa, and more preferably 0.8 kDa.

In the present invention, the size of the transporter is 10 to 50 μm, and more preferably 20 μm.

The size of the transporter is such that porous particles are utilized for delivery deep into the lungs, and generally, a size of 1 to 5 μm is the optimal size. However, since this is a size that is easily removed by macrophages, in the present invention, a transporter having a size of 20 μm, which is larger than 5 μm, can be delivered deep into the lungs without being removed by macrophages.

In the present invention, the transporter may have high porosity.

In order for the BPEI/NO to effectively deliver NO to pulmonary arterial hypertension lesions through inhalation, a stable transporter capable of reaching deep into the lungs is required. Porous particles have a low density compared to their large size, and therefore, they may be delivered deep into the lungs due to their small aerodynamic size. The PLGA is a biodegradable polymer approved by the Food and Drug Administration (FDA) and is prepared through an emulsion method. In the PLGA particle preparation process, nano-sized micropores may be formed because ammonium bicarbonate (ABC) releases carbon dioxide and ammonia gases. In addition, micro-sized pores may be formed by controlling the amount of water flowing into the PLGA particles. Therefore, inhalable porous particles may be prepared by controlling the water inflow depending on the molecular weight of BPEI/NO.

In the present invention, the transporter continuously may release NO.

As the molecular weight of the BPEI/NO decreases, the electrostatic attraction between amines and NONOates increases so that NO is released slowly. As a result, the inhalable type NO transporter may release NO at a concentration generated in the endothelial cells, thereby inducing vasodilation.

In addition, in the present invention, the transporter may not be removed by phagocytes.

As phagocytosis is inhibited, the transporter is not removed by macrophages after delivery to the lesions so that NO may be delivered for a long time.

As another aspect of the present invention, the transporter may have low cytotoxicity and excellent biocompatibility, promote cyclic guanosine monophosphate (cGMP) synthesis, and increase anti-inflammatory cytokine expression.

When the inhalable type NO transporter was co-cultured for 24 hours, no cytotoxicity was observed for smooth muscle cells and macrophages. In addition, it was confirmed that cGMP synthesis in smooth muscle cells was promoted by 0.277 nmole of NO released from an open porous NO inhaler (OPNI). The anti-inflammatory effect of the inhalable type NO transporter was analyzed by flow cytometry, and the results showed that pro-inflammatory cells were reduced and anti-inflammatory cells were increased by NO released from the OPNI. In addition, the anti-inflammatory effect of the inhalable type NO transporter was confirmed through reverse transcription quantitative polymerase chain reaction (RT-qPCR) and enzyme-linked immunosorbent assay (ELISA), and the results showed that the expression of anti-inflammatory cytokines increased in the experimental group in which macrophages were treated with the OPNI. Therefore, the prepared inhalable type NO transporter is capable of treating pulmonary arterial hypertension by continuously releasing NO.

The present invention provides a method of preparing an inhalable type NO transporter, including: (a) a step of dissolving an NO donor in an aqueous solution to prepare a first aqueous phase (W1); (b) a step of dissolving a biodegradable polymer in an organic solvent to prepare an oil phase (O); (c) a step of mixing and dispersing the first aqueous phase (W1) and the oil phase (O) to prepare a W1/O emulsion; (d) a step of mixing and dispersing the W1/O emulsion in a second aqueous phase (W2) including a surfactant to prepare a W1/O/W2 emulsion; (e) a step of stirring the W1/O/W2 emulsion to evaporate the organic solvent and then remove the surfactant and residual substances; and (f) a step of obtaining an NO transporter by freeze-drying after the removing step.

In the present invention, the NO donor may be selected from the group consisting of a BPEI/NONOates complex, a PEHA/NONOates complex, and a spermine NONOates complex, and preferably a BPEI/NONOates complex.

In addition, the biodegradable polymer of the present invention may be selected from the group consisting of hyaluronic acid, gelatin, starch, chitin, cellulose, alginate, collagen, heparin, chitosan, PLA, PGA, PLGA, PCL, PDO, PTMC, and PHA, and preferably PLGA.

The aqueous solution may be one or more selected from deionized water, a sodium acetate aqueous solution, an ammonium acetate aqueous solution, a phosphate acetate aqueous solution, a sodium citrate aqueous solution, and a sodium hydroxide aqueous solution, and more preferably deionized water.

In addition, the organic solvent may be one or more selected from the group consisting of dichloromethane, dimethyl sulfoxide, chloroform, acetone, ethyl acetate, and ethyl ether, and preferably dichloromethane.

The surfactant may one or more selected from the group consisting of polyvinyl alcohol (PVA), Tween 80, Pluronic F127, sodium carboxymethyl cellulose (NaCMC), gelatin, polysorbate, and polyethylene sorbitan monolaurate, and preferably PVA.

In addition, in Step (e), stirring may be performed for two to eight hours, and more preferably for four to six hours.

The present invention provides a method of preparing an inhalable type NO transporter including a step of preparing a transporter with a highly porous structure according to the molecular weight of an NO donor, and when BPEI/NO having a smaller molecular weight was present in the internal aqueous phase of PLGA, the movement of water increased due to a higher molar concentration difference. In addition, a larger amount of water was actually introduced into the PLGA particles.

As still another aspect of the present invention, the present invention provides a pharmaceutical composition for preventing or treating pulmonary arterial hypertension, containing the inhalable type NO transporter as an active ingredient.

The pharmaceutical composition of the present invention may include a pharmaceutically acceptable carrier or diluent, and may be formulated in the form of oral dosage forms such as powder, granules, tablets, capsules, suspensions, emulsions, syrups, and aerosols, external preparations, suppositories, and sterile injectable solutions according to conventional methods. The pharmaceutically acceptable carrier includes lactose, dextrose, sucrose, sorbitol, mannitol, xylitol, erythritol, maltitol, starch, acacia gum, alginate, gelatin, calcium phosphate, calcium silicate, cellulose, methylcellulose, microcrystalline cellulose, polyvinyl pyrrolidone, water, methyl hydroxybenzoate, propyl hydroxybenzoate, talc, magnesium stearate, mineral oils, and the like. In addition, it includes diluents or excipients such as fillers, bulking agents, binders, wetting agents, disintegrating agents, and surfactants. Oral solid preparations include tablets, pills, powder, granules, capsules, and the like, and these solid preparations may include at least one excipient, for example, starch, calcium carbonate, sucrose or lactose, gelatin, and the like, and may include lubricants such as magnesium stearate and talc. Oral liquid preparations include suspensions, solutions, emulsions, syrups, and the like, and may include diluents such as water and liquid paraffin, wetting agents, sweeteners, flavoring agents, preservatives, and the like. Parenteral preparations include sterile aqueous solutions, non-aqueous solvents, suspensions, emulsions, lyophilized preparations, and suppositories, and non-aqueous solutions and suspensions include propylene glycol, polyethylene glycol, vegetable oils such as olive oil, and injectable esters such as ethyl oleate. As a base for suppositories, Witepsol, Macrogol, Tween 61, cacao oil, laurel oil, glycerol, gelatin, or the like may be used.

The pharmaceutical composition of the present invention may be administered to mammals such as livestock and humans through various routes, for example, by oral, dermal, subcutaneous, intramuscular, intravenous, intraperitoneal, intrarectal, intrauterine, intradural or intracerebrovascular injection, and topical administration. Accordingly, the composition of the present invention may be formulated in various forms such as tablets, capsules, aqueous solutions, or suspensions. In the case of tablets for oral administration, carriers such as lactose and corn starch and lubricants such as magnesium stearate may usually be added. In the case of capsules for oral administration, lactose and/or dried corn starch may be used as diluents. When an oral aqueous suspension is required, an active ingredient may be combined with an emulsifier and/or a suspending agent. When necessary, a specific sweetener and/or flavoring agent may be added. In the case of intramuscular, intraperitoneal, subcutaneous, and intravenous administration, a sterile solution of an active ingredient is usually prepared, and the pH of the solution should be suitably adjusted and buffered. In the case of intravenous administration, the total concentration of the solute should be adjusted so that isotonicity is imparted to the formulation. The composition according to the present invention may be in the form of an aqueous solution including a pharmaceutically acceptable carrier, such as saline having a pH of 7.4. The solution may be introduced into the intramuscular bloodstream of a patient by local injection.

The dose of the active ingredient contained in the pharmaceutical composition of the present invention varies depending on the patient's condition and weight, the severity of the disease, the form of the active ingredient, and the administration route and period, and may be appropriately adjusted depending on the patient. For example, the active ingredient may be administered at a dose of 0.0001 to 300 mg/kg per day, preferably 50 to 300 mg/kg, and the administration may be administered once a day or divided into two times. In addition, the pharmaceutical composition of the present invention may include the active ingredient in an amount of 0.001% to 90% by weight based on the total weight of the composition.

In the present invention, the pharmaceutical composition may be injected using a nebulizer.

Hereinafter, one or more embodiments are described in more detail through examples. However, these examples are intended to illustrate one or more embodiments only, and the scope of the present invention is not limited to these examples.

### EXAMPLE 1: PREPARATION AND ANALYSIS OF POROUS PARTICLES ACCORDING TO MOLECULAR WEIGHT OF NO DONOR

### Preparation of Porous Particles

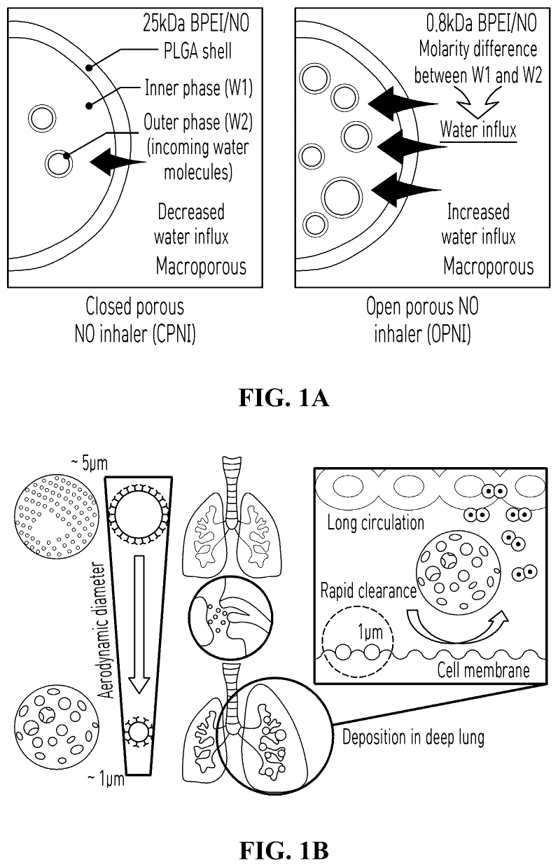

First, two types of transporters with different porosity were prepared by controlling the amount of water molecules flowing into PLGA particles using two types of NO donors according to molecular weight, as shown in FIG. 1.

Specifically, a double emulsion method was used to load hydrophilic BPEI/NO into PLGA particles. BPEI/NO dissolved in an aqueous solution (triple-distilled water) was mixed with PLGA dissolved in dichloromethane, an organic solvent, and then a W1/O emulsified solution was prepared using an ultrasonic homogenizer. Thereafter, the W1/O emulsified solution was dispersed in a solution including a surfactant (PVA) to prepare a W1/O/W2 emulsified solution using an ultrasonic homogenizer. Next, the organic solvent was evaporated by stirring the resulting solution for four to six hours. The ultimately obtained particles were washed to remove the surfactant and unloaded BPEI/NO, and then powder particles were obtained through a freeze-drying process and stored in a freezer to prevent the loss of NO.

FIG. 1B shows a schematic diagram illustrating the transpulmonary delivery efficiency of the NO transporter depending on the porous structure, indicating that the inhalable type NO transporter prepared in the present invention may continuously deliver NO to the lesions.

### Control of Porous Structure

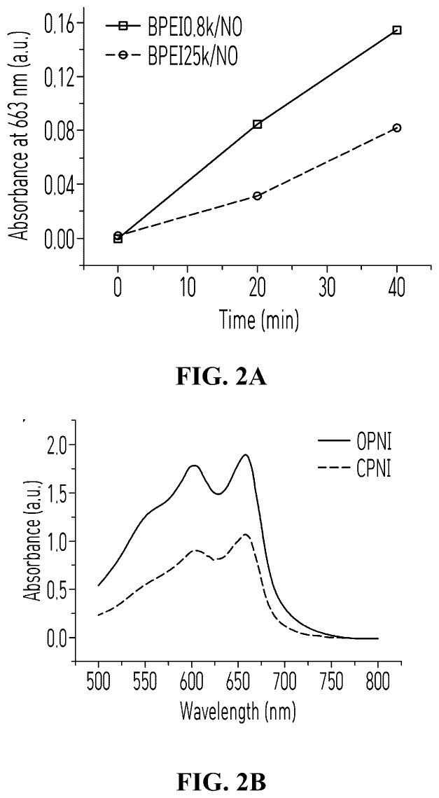

As shown in FIG. 2A, the movement of methylene blue according to the molecular weight of BPEI/NO was confirmed. As a result, it was confirmed that a larger amount of methylene blue was moved in BPEI/NO with a smaller molecular weight of 0.8 kDa.

FIG. 2B shows a graph illustrating the results of quantifying the amount of methylene blue introduced into the PLGA particles. Like the above-described results, it was confirmed that a larger amount of methylene blue was included in OPNI loaded with BPEI/NO with a smaller molecular weight.

Through these results, it was confirmed that the smaller the molecular weight of BPEI/NO, the more water was introduced into the PLGA particle, and the porous structure could be controlled.

### Porous Particle Size Analysis

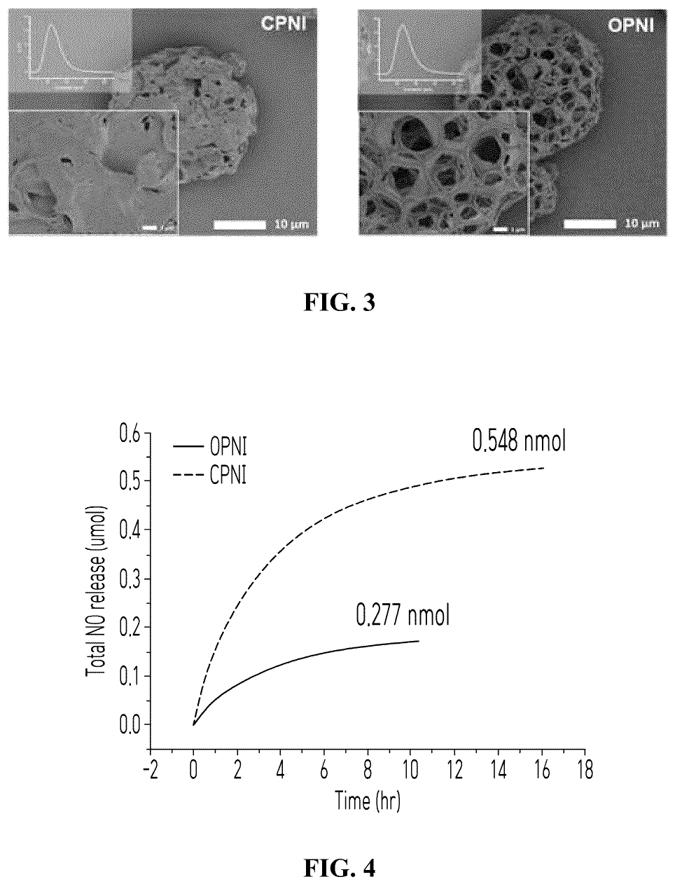

In order to confirm the size of the prepared inhalable type NO transporter, SEM analysis was performed as shown in FIG. 3. It was confirmed that both types of transporters with different porosity had a size of about 20 μm and that particles with high porosity were formed as the water inflow increased. In addition, it was confirmed that low-molecular weight BPNO exhibited the effect of continuously releasing NO as the electrostatic attraction and stability increased.

### EXAMPLE 2: ANALYSIS OF NO RELEASE BEHAVIOR OF INHALABLE TYPE NO TRANSPORTER

The NO release behavior of the prepared inhalable type NO transporter was analyzed using an NO analyzer (see FIG. 3). Under biomimetic conditions, it was confirmed that the OPNI loaded with 0.8 kDa BPEI/NO continuously released 0.277 nmole of NO for about 10 hours.

Through these results, it was confirmed that as the molecular weight of BPEI/NO decreased, the electrostatic attraction between amines and NONOates increased, and NO was released slowly, and as a result, the inhalable type NO transporter may release NO at a concentration generated in the endothelial cells, thereby inducing vasodilation.

### EXAMPLE 3: ANALYSIS OF DELIVERY EFFICIENCY OF INHALABLE TYPE NO TRANSPORTER THROUGH NEBULIZER

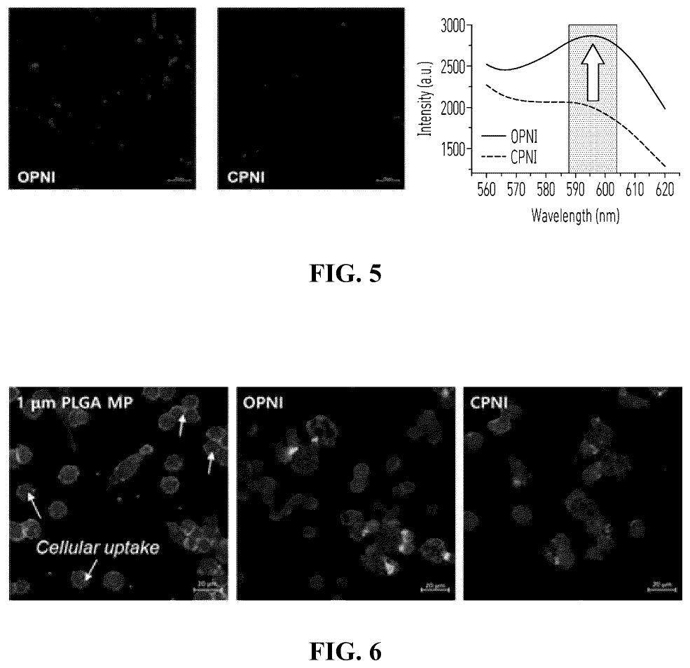

As shown in FIG. 5, the delivery efficiency of the prepared inhalable type NO transporter through a nebulizer was analyzed in a respiratory model after Nile red was loaded. The particles that reached the lungs were visually confirmed through fluorescence microscopy images. When the amount of delivered particles was quantitatively analyzed, it was confirmed that a larger amount of OPNI was delivered to the lungs than that of CPNI.

As a result, it was confirmed that OPNI with high porosity can be delivered deep into the lungs due to its small aerodynamic size.

### Example 4: ANALYSIS OF PHAGOCYTOSIS INHIBITION ACCORDING TO THE DIAMETER OF INHALABLE TYPE NO TRANSPORTER

As shown in FIG. 6, it was confirmed that the prepared inhalable type NO transporter was not removed by macrophages.

Specifically, in order to analyze the phagocytosis inhibition according to the particle diameter, particles with a diameter of 1 um were prepared as a control. After co-culturing the macrophages and particles for four hours, fluorescence microscope images were analyzed, and as a result, it was confirmed that, unlike the control, the prepared inhalable type NO transporter did not undergo phagocytosis by macrophages due to the high porosity and large size of the particles.

Through these results, it was confirmed that the inhalable type NO transporter may deliver NO for a long time without being removed by macrophages after being delivered to the lesions.

### EXAMPLE 5: ANALYSIS OF THERAPEUTIC FUNCTION OF INHALABLE TYPE NO TRANSPORTER FOR PULMONARY ARTERIAL HYPERTENSION

The therapeutic effect of the prepared inhalable type NO transporter for pulmonary arterial hypertension was analyzed as shown in FIGS. 6 to 10.

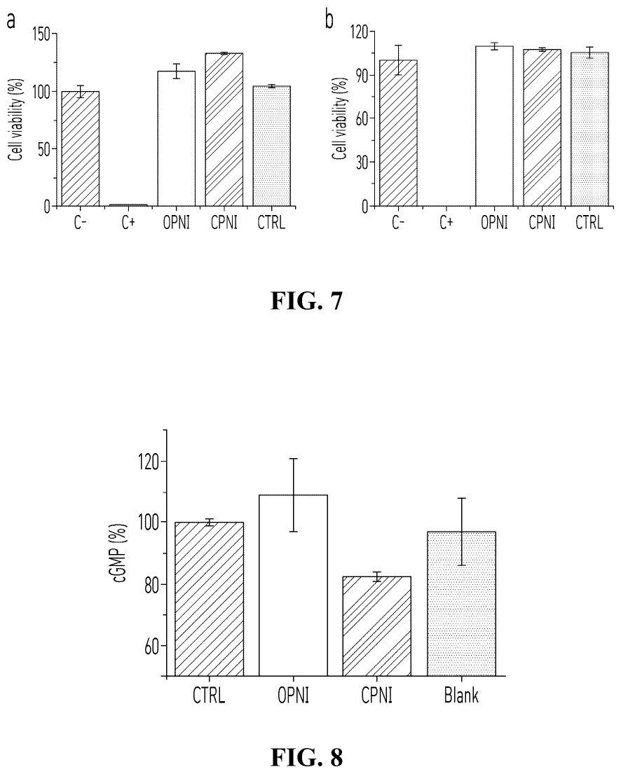

Specifically, the biocompatibility of the prepared inhalable type NO transporter was confirmed. FIG. 7 shows that when the inhalable type NO transporter was co-cultured for 24 hours, no cytotoxicity was observed for smooth muscle cells and macrophages.

As shown in FIG. 8, as a result of confirming the improvement of cGMP synthesis by the prepared inhalable type NO transporter, it was confirmed that cGMP synthesis in smooth muscle cells was promoted by 0.277 nmole of NO released from OPNI.

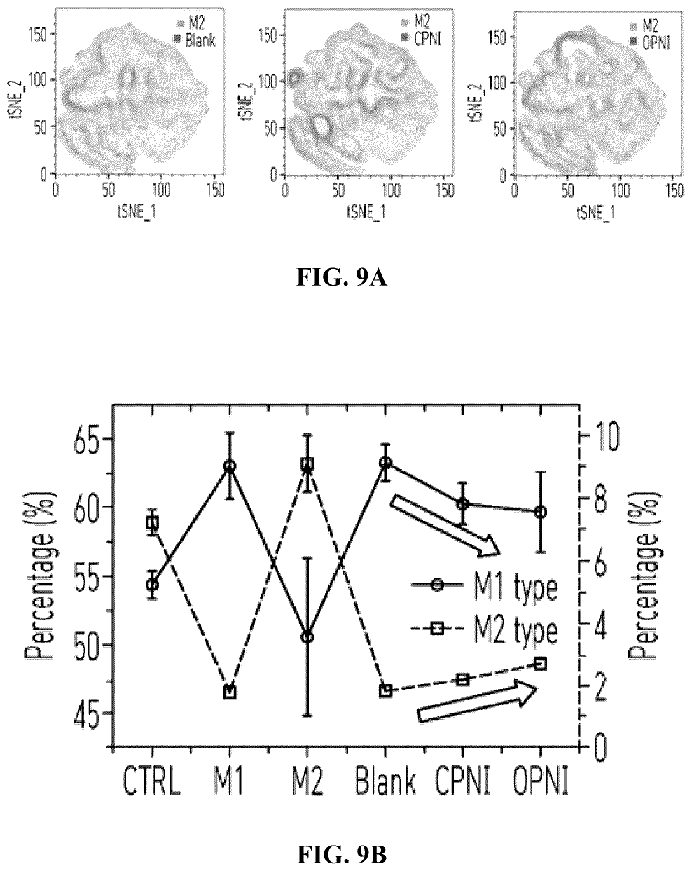

As shown in FIGS. 9A-9B, the anti-inflammatory effect of the inhalable type NO transporter was analyzed by flow cytometry. As a result, it was confirmed that unlike CPNI, the pro-inflammatory cells decreased and the anti-inflammatory cells increased due to the NO released from OPNI.

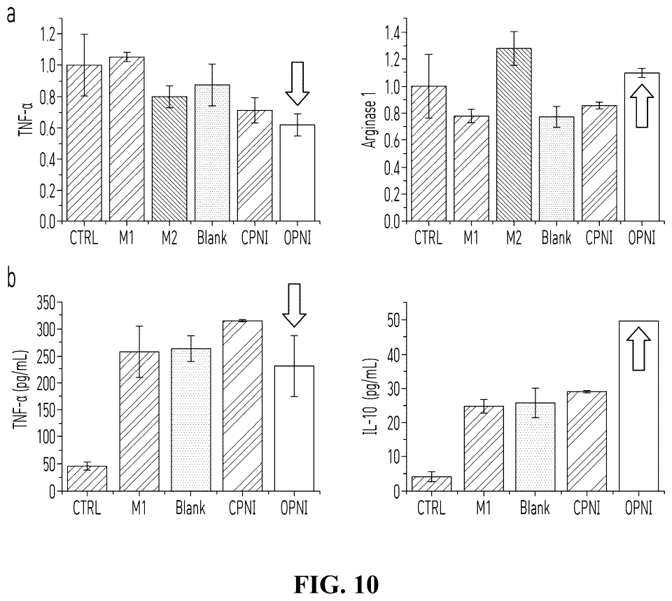

(FIG. 10) The anti-inflammatory effect of the inhalable type NO transporter was confirmed through RT-qPCR and ELISA, and as a result, it was found that the expression of anti-inflammatory cytokines increased in the experimental group in which macrophages were treated with OPNI.

Through these results, it was confirmed that the prepared inhalable type NO transporter may treat pulmonary arterial hypertension by continuously releasing NO.

Therefore, the effects of the inhalable type NO transporter, such as inducing vasodilation, NO delivery efficiency, and phagocytosis inhibition, were confirmed, and the possibility of using as a therapeutic agent for pulmonary arterial hypertension was confirmed.

Conventional methods for treating pulmonary arterial hypertension using NO have many limitations in clinical application due to the complicated inhalation system, but the inhalable type NO transporter prepared in the present invention can be expected to exhibit a treatment effect simply and safely, because it delivers NO-releasing particles through a nebulizer. In addition, while previous inhalation systems were accompanied by equipment for monitoring NO, the inhalable type NO transporter according to the present invention can continuously deliver NO at low concentrations, so it has excellent versatility and is significant in research and industry.

The present invention has been described with reference to preferred examples thereof. Those skilled in the art will understand that the present invention may be implemented in modified forms without departing from the essential characteristics of the present invention. Therefore, the disclosed examples should be considered from an illustrative perspective rather than a restrictive perspective. The scope of the present invention is indicated by the claims, not the foregoing description, and all differences within the scope equivalent thereto should be construed as being included in the present invention.

What is claimed is:

1. An inhalable type nitric oxide transporter comprising a biodegradable polymer shell including a nitric oxide donor therein.

2. The inhalable type nitric oxide transporter according to claim 1, wherein the nitric oxide donor is selected from the group consisting of a branched polyethyleneimine (BPEI)/NONOates complex, a pentaethylenehexamine (PEHA)/NONOates complex, and a spermine NONOates complex.

3. The inhalable type nitric oxide transporter according to claim 1, wherein the biodegradable polymer is selected from the group consisting of hyaluronic acid, gelatin, starch, chitin, cellulose, alginate, collagen, heparin, chitosan, polylactic acid (PLA), polyglycolic acid (PGA), poly(lactic-co-glycolic acid) (PLGA), polycaprolactone (PCL), polydioxanone (PDO), poly(trimethylene carbonate) (PTMC), and polyhydroxyalkanoate (PHA).

4. The inhalable type nitric oxide transporter according to claim 1, wherein the molecular weight of the BPEI/NONOates complex is 0.1 to 3.0 kDa.

5. The inhalable type nitric oxide transporter according to claim 1, wherein the size of the transporter is 10 to 50 μm.

6. The inhalable type nitric oxide transporter according to claim 1, wherein the transporter has high porosity.

7. The inhalable type nitric oxide transporter according to claim 1, wherein the transporter continuously releases nitric oxide.

8. The inhalable type nitric oxide transporter according to claim 1, wherein the transporter is not removed by phagocytes.

9. The inhalable type nitric oxide transporter according to claim 1, wherein the transporter has low cytotoxicity and excellent biocompatibility.

10. The inhalable type nitric oxide transporter according to claim 1, wherein the transporter promotes cyclic guanosine monophosphate (cGMP) synthesis and increases anti-inflammatory cytokine expression.

11. A method of preparing an inhalable type nitric oxide transporter, comprising:
(a) a step of dissolving a nitric oxide donor in an aqueous solution to prepare a first aqueous phase (W1);
(b) a step of dissolving a biodegradable polymer in an organic solvent to prepare an oil phase (O);
(c) a step of mixing and dispersing the first aqueous phase (W1) and the oil phase (O) to prepare a W1/O emulsion;
(d) a step of mixing and dispersing the W1/O emulsion in a second aqueous phase (W2) including a surfactant to prepare a W1/O/W2 emulsion;
(e) a step of stirring the W1/O/W2 emulsion to evaporate the organic solvent and then remove the surfactant and residual substances; and
(f) a step of obtaining a nitric oxide transporter by freeze-drying after the removing step.

12. The method of preparing the inhalable type nitric oxide transporter according to claim 11, wherein the nitric oxide donor is selected from the group consisting of a branched polyethyleneimine (BPEI/NONOates complex, a pentaethylenehexamine (PEHA)/NONOates complex, and a spermine NONOates complex.

13. The method of preparing the inhalable type nitric oxide transporter according to claim 11, wherein the biodegradable polymer is selected from the group consisting of hyaluronic acid, gelatin, starch, chitin, cellulose, alginate, collagen, heparin, chitosan, polylactic acid (PLA), polyglycolic acid (PGA), poly(lactic-co-glycolic acid) (PLGA), polycaprolactone (PCL), polydioxanone (PDO), poly(trimethylene carbonate) (PTMC), and polyhydroxyalkanoate (PHA).

14. The method of preparing the inhalable type nitric oxide transporter according to claim 11, wherein the aqueous solution is one or more selected from deionized water, a sodium acetate aqueous solution, an ammonium acetate aqueous solution, a phosphate acetate aqueous solution, a sodium citrate aqueous solution, and a sodium hydroxide aqueous solution.

15. The method of preparing the inhalable type nitric oxide transporter according to claim 11, wherein the organic solvent is one or more selected from the group consisting of dichloromethane, dimethyl sulfoxide, chloroform, acetone, ethyl acetate, and ethyl ether.

16. The method of preparing the inhalable type nitric oxide transporter according to claim 11, wherein the surfactant is one or more selected from the group consisting of polyvinyl alcohol (PVA), Tween 80, Pluronic F127, sodium carboxymethyl cellulose (NaCMC), gelatin, polysorbate, and polyethylene sorbitan monolaurate.

17. The method of preparing the inhalable type nitric oxide transporter according to claim 11, wherein, in Step (e), stirring is performed for two to eight hours.

18. A pharmaceutical composition for preventing or treating pulmonary arterial hypertension, comprising the inhalable type nitric oxide transporter according to claim 1 as an active ingredient.

19. The pharmaceutical composition for preventing or treating pulmonary arterial hypertension according to claim 18, wherein the transporter is contained in an amount of 0.0001% to 1% by weight based on the total weight of the composition.

20. The pharmaceutical composition for preventing or treating pulmonary arterial hypertension according to claim 18, wherein the pharmaceutical composition is injected using a nebulizer.
